(19) United States
(12) Patent Application Publication
(10) Pub. No.: US 2014/0099352 A1
(43) Pub. Date: Apr. 10, 2014

(54) Compositions, Structures and Methods for Neural Regeneration

(71) Applicant: Robert G. Matheny, Norcross, GA (US)

(72) Inventor: Robert G. Matheny, Norcross, GA (US)

(21) Appl. No.: 14/031,189

(22) Filed: Sep. 19, 2013

Related U.S. Application Data
(60) Provisional application No. 61/711,018, filed on Oct. 8, 2012.

(51) Int. Cl. A61L 27/36; A61K 31/722; A61L 27/54; A61K 35/12

(52) U.S. Cl. CPC A61L 27/3633; A61K 35/12; A61K 31/722; A61L 27/3675; A61L 27/54
USPC 424/423; 424/572

(57) ABSTRACT

A nerve regeneration device comprising a support structure having an outer surface and a plurality of conduits extending therethrough, the support structure comprising a first extracellular matrix (ECM) material from a mammalian tissue source, the support structure outer layer including at least a first layer comprising a first ECM composition having at least a second ECM material from a mammalian tissue source. When the nerve regeneration device is deployed proximate damaged neural tissue, the device induces modulated healing of the damaged tissue.

### CROSS-REFERENCES TO RELATED APPLICATIONS

This application claims the benefit of U.S. Application Nos. 61/711,018, filed on Oct. 8, 2012.

### FIELD OF THE INVENTION

The present invention relates to compositions and methods for promoting nerve growth and/or regeneration. More particularly, the present invention relates to extracellular matrix (ECM) based compositions, structures and methods for promoting nerve growth and/or regeneration.

### BACKGROUND OF THE INVENTION

While soft tissues (e.g., muscle and skin) and bone possess considerable capacity for recovery after injury, inadequate nerve repair frequently limits the extent to which normal function is regained. In the peripheral nervous system (PNS), nerves are often able to regenerate on their own, if the injury is small enough.

Larger injuries can be effectively treated surgically, either by direct reconnection of damaged nerve ends with the nerve sheath previously employed by the axons to reach their destination or with grafts harvested from elsewhere in the body. However, clinical functional recovery rates generally approach only 80% following nerve graft, and the procedure has the additional disadvantage of requiring two surgeries.

An alternative approach that is often employed to repair nerve damage is to provide an artificial conduit to facilitate axonal growth across a nerve gap, such as the NeuraGen® collagen tube. However, this treatment is typically reserved for small defects (e.g., several millimeters).

In the central nervous system (CNS), nerves have a limited capability to regenerate upon injury. This limited regenerative ability can be attributed to several factors. For example, injury to CNS axons often elicits detrimental inflammatory responses, which are followed by secondary degeneration of the nervous tissues. In addition, regeneration of injured axons is believed to be impeded by the presence or up-regulation of various nerve outgrowth inhibitors, including myelin-associated inhibitors and repulsive axon-guidance molecules, and the absence or down-regulation of factors that promote nerve outgrowth and cell survival, including neurotrophic factors.

Known inhibitors of CNS axon regeneration include, for example, ephrin-B3 and Nogo, and, as discussed in detail herein, chondroitin sulphate proteoglycans (CSPGs) in the chronic postinjury phase.

Ephrin-B3 (EFNB3) is a 340-amino acid, transmembrane protein that belongs to the class of ephrin-B (EFNB) ligands. The EFNB ligands bind Eph-family receptor protein tyrosine kinases, such as EphA4.

EFNB3-EphA4 signaling is believed to play a role in the inhibitory activity of CNS myelin preparations. Several reports indicate that EphA4 accumulates in proximal axon stumps and EphA4 ligands, EFNB2 and EFNB3, which are markedly up-regulated in astrocytes in the glial scar. These events are thought to lead to retraction of corticospinal axons and inhibition of their regeneration.

Nogo also inhibits nerve regeneration via interactions with its receptor (NgR). Members of the tumor necrosis factor receptor (TNFR) superfamily have particularly been shown to be involved in NgR-mediated inhibition of nerve regeneration through promotion of inflammatory responses.

As stated above, chondroitin sulphate proteoglycans (CSPGs) can, and in most instances will, inhibit nerve regeneration in the chronic postinjury phase. CSPGs are components of the extracellular matrix (ECM) and are naturally occurring throughout the body.

During development, CSPGs play a vital role by forming boundaries that guide migrating neuronal cells to appropriate destinations. Although the general consensus is that CSPGs inhibit nerve cell regeneration and axonal growth by virtue of the substantially increased levels of CSPGs present at glial scars, see, e.g., Properzi, et al., Chondroitin Sulfate Proteoglycans in the Central Nervous System: Changes and Synthesis After Injury, Biochem Soc Trans, vol. 31, pp. 335-336 (2003), it has been found that CSPGs are required at the early stages of recovery, i.e. the acute phase, (or after the scar tissue is removed) to promote and/or facilitate nerve cell regeneration and axonal growth, see Rolls, et al., Two Faces of Chondroitin Sulfate Proteoglycan in Spinal Cord Repair: A Role in Microglia/Macrophage Activation, PLoS Med, vol. 5(8), pp. 172-186 (2008); and Silver, et al., Regeneration Beyond the Glial Scar, Nat. Rev. Neurosci., vol. 5, pp. 146-156 (2004).

Despite medical advancements to restore nerve function in the CNS via the delivery of selective molecules that impede the presence or up-regulation of various nerve outgrowth inhibitors and/or promote nerve outgrowth and cell survival, there is currently no effective treatment available to completely restore nerve function in the CNS. Rehabilitation, in which patients train remaining nerves to compensate for loss due to injury, remains the mainstay of therapy.

It would thus be desirous to provide improved compositions and methods that suppress inhibitory nerve regeneration mechanisms and/or enhance neurotrophic nerve regeneration mechanisms following CNS injury to overcome the limited ability of the CNS to recover from injury.

It is therefore an object of the present invention to provide extracellular matrix (ECM) based compositions, structures and methods that effectively suppress inhibitory nerve regeneration mechanisms and enhance neurotrophic nerve regeneration mechanisms following PNS and CNS injury.

It is another object of the present invention to provide ECM based compositions, structures and methods that inhibit Wallerian degeneration mechanisms.

It is another object of the present invention to provide ECM based compositions, structures and methods that induce modulated healing of damaged and/or diseased neural tissue.

It is another object of the present invention to provide ECM based compositions, structures and methods that induce regeneration of neural tissue and structures with site-specific functional properties.

It is another object of the present invention to provide ECM based compositions, structures and methods that modulate the inflammatory phase (e.g., platelet or fibrin deposition) at the beginning of the tissue healing process.

It is another object of the present invention to provide ECM based compositions, structures and methods that induce host tissue proliferation and bioremodeling, including neovascularization.

### SUMMARY OF THE INVENTION

The present invention is directed to ECM based compositions, structures and methods that modulate healing of damaged neural tissue and promote nerve growth and/or regeneration.

In a preferred embodiment of the invention, the ECM based structures, i.e. ECM nerve regeneration members, include an ECM core member or structure, which can comprise various shapes and configurations.

In some embodiments, the ECM core member comprises a tubular (or cylindrical shaped) core member having a plurality of conduits extending therethrough.

In some embodiments of the invention, the ECM based structures include an ECM core member comprising an ECM material and at least one ECM composition layer that is designed and/or configured to be disposed on the outer surface of the core member.

In some embodiments, the ECM core member has a tubular shape.

In some embodiments, the ECM composition layer comprises an ECM composition coating. In some embodiments, the ECM composition layer comprises a plurality of ECM composition coatings.

In some embodiments, the ECM composition layer comprises an ECM composition sheet member. In some embodiments, the ECM composition layer comprises a plurality of ECM composition sheet members.

In some embodiments, the ECM composition layer comprises at least one ECM composition coating and at least one ECM composition sheet member.

In a preferred embodiment, the ECM compositions include at least one ECM material. According to the invention, the ECM material can be derived from various mammalian tissue sources, including, without limitation, the small intestine, large intestine, stomach, lung, liver, kidney, pancreas, placenta, heart, bladder and prostate.

In some embodiments, the ECM compositions further include one or more additional biologically active components to facilitate the treatment of damaged tissue and/or the tissue regenerative process.

In some embodiments, the ECM compositions thus include at least one pharmacological agent or composition, which can comprise, without limitation, antibiotics or antifungal agents, anti-viral agents, anti-pain agents, anesthetics, analgesics, steroidal anti-inflammatories, non-steroidal anti-inflammatories, anti-neoplastics, anti-spasmodics, modulators of cell-extracellular matrix interactions, proteins, hormones, enzymes and enzyme inhibitors, anticoagulants and/or antithrombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

In some embodiments of the invention, the pharmacological agent specifically comprises an anti-inflammatory agent or composition.

In some embodiments of the invention, the biologically active component comprises a statin, which can comprise, without limitation, atorvastatin, cerivastatin, fluvastatin, lovastatin, mevastatin, pitavastatin, pravastatin, rosuvastatin, and simvastatin.

In some embodiments, the biologically active component comprises chitosan.

In some embodiments, the biologically active agent comprises a growth factor.

In some embodiments, the biologically active component comprises a cell.

In some embodiments, the biologically active component comprises a protein.

In some embodiments of the invention, the ECM compositions are formulated to facilitate injection of the ECM compositions to damaged or diseased tissue (i.e. injectable ECM compositions).

According to the invention, upon deployment of an ECM nerve regeneration member or ECM composition of the invention in a damaged or resected neural pathway, modulated healing of neural tissue, including suppression of inhibitory nerve regeneration mechanisms and enhancement of nerve regeneration mechanisms, is effectuated.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1A is an illustration of neural tissue, e.g., spinal cord, having a region of scar tissue;

FIG. 1B is a side elevational view of an ECM nerve regeneration member inserted in the neural tissue shown in FIG. 1A after the scar tissue was debrided, in accordance with the invention;

FIG. 1C is a side elevational view of the ECM nerve regeneration member inserted in the neural tissue, as shown in FIG. 1B, and after the ECM nerve regeneration member and a region of the neural tissue is wrapped with an ECM composition sheet, in accordance with the invention;

FIG. 2A is another illustration of neural tissue having a region of scar tissue;

FIG. 2B is a side elevational view of an ECM nerve regeneration member inserted in the neural tissue shown in FIG. 2A after the scar tissue was debrided, in accordance with the invention;

FIG. 2C is a side elevational view of the ECM nerve regeneration member inserted in the neural tissue, as shown in FIG. 2B, and after the ECM nerve regeneration member and a region of the neural tissue is wrapped with an ECM composition sheet, in accordance with the invention;

FIG. 3 is a side elevational view of an ECM composition sheet disposed over a region of neural tissue after scar tissue thereon was debrided, in accordance with the invention; and

FIG. 4 is a perspective view of another embodiment of an ECM core structure having a plurality of conduits extending therethrough, in accordance with the invention.

### DETAILED DESCRIPTION OF THE PREFERRED EMBODIMENT

Before describing the present invention in detail, it is to be understood that this invention is not limited to particularly exemplified apparatus, systems, structures or methods as such may, of course, vary. Thus, although a number of apparatus, systems and methods similar or equivalent to those described herein can be used in the practice of the present invention, the preferred apparatus, systems, structures and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “an active agent” includes two or more such agents and the like.

### DEFINITIONS

The term “nerve”, as used herein, means and includes both nonfascicular and polyfascicular nerves.

The term “glial cell”, as used herein, means and includes a non-neuronal cell that provides support and nutrition, maintains homeostasis, forms myelin, and/or participates in signal transmission in the nervous system. Glial cells include, but are not limited to microglia, macroglia, astrocytes, oligodendrocytes, radial cells, and ependymal cells in the CNS, and Schwann cells and satellite cells in the PNS.

Astrocytes are the most abundant type of glial cell. Astrocytes regulate the external chemical environment of neurons by removing excess ions, notably potassium, and recycling neurotransmitters released during synaptic transmission. Astrocytes also form much of the blood-brain barrier.

Astrocytes can also regulate vasoconstriction and vasodilation by producing substances, such as arachidonic acid that generate vasoactive metabolites. In addition, astrocytes form gap junctions with other astrocytes, which permit signaling between the cells.

The term “microglia”, as used herein, means and includes specialized macrophages that are capable of phagocytosis. Although not technically glia (because they are derived from monocytes rather than ectodermal tissue), they are commonly categorized as such because of their supportive role to neurons.

The term “oligodendrocytes”, as used herein, means a glial cell that facilitates the formation of myelin, i.e. an insulating layer around CNS axons.

The term “Schwann cell”, as used herein, means a glial cell that wraps around the nerve fiber in the peripheral nervous system, and forms the myelin sheaths of peripheral axons. In the PNS, Schwann cells play a role similar to that of oligodendrocytes in the CNS, providing myelination to PNS axons. Schwann cells also possess the capacity to present antigens to T-lymphocytes, and can be myelinating or non-myelinating.

The terms “extracellular matrix”, “extracellular matrix material” and “ECM material” are used interchangeably herein, and mean a collagen-rich substance that is found in between cells in animal tissue and serves as a structural element in tissues. It typically comprises a complex mixture of polysaccharides and proteins secreted by cells. The extracellular matrix can be isolated and treated in a variety of ways. Extracellular matrix material (ECM) can be isolated from small intestine submucosa, stomach submucosa, urinary bladder submucosa, tissue mucosa, dura mater, liver basement membrane, pericardium or other tissues. Following isolation and treatment, it is commonly referred to as extracellular matrix or ECM material.

The terms “pharmacological agent”, “pharmaceutical agent”, “agent”, “active agent”, “drug” and “active agent formulation” are used interchangeably herein, and mean and include an agent, drug, compound, composition of matter or mixture thereof, including its formulation, which provides some therapeutic, often beneficial, effect. This includes any physiologically or pharmacologically active substance that produces a localized or systemic effect or effects in animals, including warm blooded mammals, humans and primates; avians; domestic household or farm animals, such as cats, dogs, sheep, goats, cattle, horses and pigs; laboratory animals, such as mice, rats and guinea pigs; fish; reptiles; zoo and wild animals; and the like.

The terms “pharmacological agent”, “pharmaceutical agent”, “agent”, “active agent”, “drug” and “active agent formulation” thus mean and include, without limitation, antibiotics, anti-viral agents, analgesics, steroidal anti-inflammatories, non-steroidal anti-inflammatories, anti-neoplastics, anti-spasmodics, modulators of cell-extracellular matrix interactions, proteins, hormones, enzymes and enzyme inhibitors, anticoagulants and/or antithrombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

The terms “anti-inflammatory” and “anti-inflammatory agent” are also used interchangeably herein, and mean and include a “pharmacological agent” and/or “active agent formulation”, which, when a therapeutically effective amount is administered to a subject, prevents or treats bodily tissue inflammation i.e. the protective tissue response to injury or destruction of tissues, which serves to destroy, dilute, or wall off both the injurious agent and the injured tissues. Anti-inflammatory agents thus include, without limitation, alclofenac, alclometasone dipropionate, algestone acetonide, alpha amylase, amcinafal, amcinafide, amfenac sodium, amprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, decanoate, deflazacort, delatestryl, depo-testosterone, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, mesterolone, methandrostenolone, methenolone, methenolone acetate, methylprednisolone suleptanate, morniflumate, nabumetone, nandrolone, naproxen, naproxen sodium, naproxol, nimazone, olsalazine sodium, orgotein, orpanoxin, oxandrolane, oxaprozin, oxyphenbutazone, oxymetholone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, stanozolol, sudoxicam, sulindac, suprofen, talmetacin, talniflumate, talosalate, tebufelone, tenidap, tenidap sodium, tenoxicam, tesicam, tesimide, testosterone, testosterone blends, tetrydamine, tiopinac, tixocortol pivalate, tolmetin, tolmetin sodium, triclonide, triflumidate, zidometacin, and zomepirac sodium.

The term “chitosan”, as used herein, means and includes the family of linear polysaccharides consisting of varying amounts of β (1→4) linked residues of N-acetyl-2 amino-2-deoxy-D-glucose and 2-amino-2-deoxy-Dglucose residues, and all derivatives thereof.

The term “pharmacological composition”, as used herein, means and includes a composition comprising a “pharmacological agent” and/or an “extracellular matrix material” and/or a “pharmacological agent formulation” and/or any additional agent or component identified herein.

The term “therapeutically effective”, as used herein, means that the amount of an ECM composition of the invention that is administered to neural tissue is of sufficient quantity to induce modulated healing of damaged or diseased neural tissue.

The terms “delivery” and “administration” are used interchangeably herein, and mean and include providing an “ECM composition” or “ECM nerve regeneration member” of the invention to a treatment site, e.g. proximate neural tissue, through any method appropriate to deliver the functional composition or member to the treatment site.

The terms “patient” and “subject” are used interchangeably herein, and mean and include warm blooded mammals, humans and primates; avians; domestic household or farm animals, such as cats, dogs, sheep, goats, cattle, horses and pigs; laboratory animals, such as mice, rats and guinea pigs; fish; reptiles; zoo and wild animals; and the like.

The term “comprise” and variations of the term, such as “comprising” and “comprises,” means “including, but not limited to” and is not intended to exclude, for example, other additives, components, integers or steps.

The following disclosure is provided to further explain in an enabling fashion the best modes of performing one or more embodiments of the present invention. The disclosure is further offered to enhance an understanding and appreciation for the inventive principles and advantages thereof, rather than to limit in any manner the invention. The invention is defined solely by the appended claims including any amendments made during the pendency of this application and all equivalents of those claims as issued.

As indicated above, the present invention is directed to extracellular matrix (ECM) based compositions, structures and methods that modulate healing of damaged neural tissue. The phrase “modulated healing”, as used herein, includes the modulation (or regulation) of several different biologic mechanisms relating to neural tissue repair and regeneration, including, without limitation, modulation of (i) Wallerian degeneration mechanisms, (ii) host tissue proliferation and bioremodeling, (iii) connective fibrous tissue production and function, (iv) fibrin deposition, (v) platelet activation and attachment, and (vi) inflammatory phases and responses, and their interplay with each other.

In a preferred embodiment of the invention, the ECM based structures, i.e. ECM nerve regeneration members, include an ECM core member or structure, which can comprise various shapes and configurations.

In some embodiments, the ECM core member comprises a tubular (or cylindrical shaped) core member having a plurality of conduits extending therethrough.

In some embodiments of the invention, the ECM based structures include a tubular shaped ECM core member that includes an ECM material and at least one ECM composition layer that is designed and/or configured to be disposed on the outer surface of the core member.

In some embodiments, the ECM composition layer comprises an ECM composition coating. In some embodiments, the ECM composition layer comprises a plurality of ECM composition coatings.

In some embodiments, the ECM composition layer comprises an ECM composition sheet member. In some embodiments, the ECM composition layer comprises a plurality of ECM composition sheet members.

In some embodiments, the ECM composition layer comprises at least one ECM composition coating and at least one ECM composition sheet member.

According to the invention, upon deployment of an ECM nerve regeneration member of the invention to damaged neural tissue or in a resected neural pathway, modulated healing, including the regeneration of neural tissue and structures with site-specific functional properties, is effectuated.

As is well known in the art, regeneration of neural tissue in the PNS after injury comprises several related sequence of events. After injury, the PNS immediately elicits the migration of phagocytes to the lesion site in order to clear away debris, such as damaged tissue.

Thereafter, axonal sprouts form at the proximal stump and grow until they enter the distal stump. The growth of the sprouts are governed by chemotactic factors secreted from the Schwann cells (neurolemmocytes).

The proximal end also swells and experiences some retrograde degeneration, but once the debris is cleared, it begins to sprout axons and the presence of growth cones can be detected. The proximal axons are able to regrow as long as the cell body is intact, and they have made contact with the Schwann cells in the endoneurial channel.

The distal segment, however, experiences Wallerian degeneration within hours of the injury; the axons and myelin degenerate, but the endoneurium remains. In the later stages of regeneration the remaining endoneurial tube directs axon growth back to the correct targets.

During Wallerian degeneration, Schwann cells grow in ordered columns along the endoneurial tube, creating a band of Biingner (boB) that protects and preserves the endoneurial channel. Further, macrophages and Schwann cells release neurotrophic factors and cytokines that enhance regeneration of neural tissue.

Additional prominent proteins that are expressed in PNS regeneration, include collagen I &II, laminin gamma-1, and fibronectine. Indeed, increased levels of the noted proteins have been found in the guide and proximal sections of a regenerating nerve. The distal segment also possessed early increases of laminin gamma-1 and fibronectine.

Collagen I &II, laminin gamma-1 and fibronectine are also major constituents of the ECM compositions of the invention and, hence, when administered to (or disposed proximate to) damaged neural tissue enhance the regeneration of the damaged tissue.

As indicated above, unlike PNS injury, CNS injury is not followed by extensive regeneration. Neural regeneration is limited by the inhibitory influences of the glial and extracellular environment. The hostile, growth inhibiting environment is, in part, created by the migration of myelin-associated inhibitors, astrocytes, oligodendrocytes, oligodendrocyte precursors, and microglia.

Neural regeneration of CNS tissue; particularly, the meninges, can, however, be induced and/or enhanced by the ECM compositions of the invention. The meninges is the system of membranes that envelops the CNS. The primary function of the meninges and of the cerebrospinal fluid is to protect the central nervous system. In mammals, the meninges consist of three layers: the dura mater, the arachnoid mater, and the pia mater.

The dura mater is a thick, durable, fibrous connective tissue similar to cartilage, which ECM has shown to differentiate into during the regeneration process.

The middle element of the meninges is the arachnoid mater, so named because of its spider web-like appearance. The arachnoid mater provides a cushioning effect for the CNS. The ECM's natural matrix proteins minor the arachnoid structure.

The pia mater is the meningeal envelope that adheres to the surface of the spinal cord. The pia mater is pierced by blood vessels that travel to the brain and spinal cord. Its capillaries are responsible for nourishing the brain.

The subarachnoid space is the space that normally exists between the arachnoid and the pia mater, which is filled with cerebrospinal fluid (CSF) and blood vessels. Normally, the dura mater is attached to the bones of the vertebral canal in the spinal cord.

The arachnoid is attached to the dura mater, while the pia mater is attached to the CNS tissue. CNS injury often presents a separation between the dura mater and the arachnoid.

The ECM compositions of the invention have, however, demonstrated the ability in angiogenesis to promote the regeneration of neural tissue by, among other things, establishing the connection between regenerated neural tissue and blood supplies.

As indicated above, in a preferred embodiment, the ECM compositions (and/or ECM core members) of the invention include at least one extracellular matrix (hereinafter “ECM material”). According to the invention, the ECM material can be derived from various mammalian tissue sources and methods for preparing same, such as disclosed in U.S. Pat. Nos. 7,550,004, 7,244,444, 6,379,710, 6,358,284, 6,206,931, 5,733,337 and 4,902,508 and U.S. application Ser. No. 12/707,427; which are incorporated by reference herein in their entirety. The mammalian tissue sources include, without limitation, the small intestine, large intestine, stomach, lung, liver, kidney, pancreas, placenta, heart, bladder, prostate, tissue surrounding growing enamel, tissue surrounding growing bone, and any fetal tissue from any mammalian organ.

As is well known in the art, the urinary bladder submucosa is an extracellular matrix that has the tunica mucosa (which includes the transitional epithelial layer and the tunica propria), a submucosal layer, three layers of muscularis, and the adventitia (a loose connective tissue layer). This general configuration is true also for small intestine submucosa (SIS) and stomach submucosa (SS).

Other tissues, such as the liver and pancreas have ECM material called basement membrane. Basement membrane generally does not demonstrate the kind of tensile strength found in submucosa. However, other useful properties may be opportunistically employed from the ECM material of such tissues as the liver, pancreas, placenta and lung tissues; all of which have either a basement membrane or interstitial membrane (as with the lung). For example, pancreatic extracellular membrane supports beta islet cells that are critical to pancreatic function. Also, for example, the liver is one tissue known to be able to regenerate itself and therefore special qualities may be present in the liver basement membrane that help facilitate that process. The ECM material surrounding developing tooth enamel and developing bone also have particular advantages over other matrices in that they support the growth and differentiation of the hard tissues of bone and enamel.

According to the invention, the ECM material can be used in whole or in part, so that, for example, an ECM material can contain just the basement membrane (or transitional epithelial layer) with the subadjacent tunica propria, the tunica submucosa, tunica muscularis, and tunica serosa. The ECM material component of the composition can contain any or all of these layers, and thus could conceivably contain only the basement membrane portion, excluding the submucosa. However, generally, and especially since the submucosa is thought to contain and support the active growth factors and other proteins necessary for in vivo tissue regeneration, the ECM or matrix composition from any given source will contain the active extracellular matrix portions that support cell development and differentiation and tissue regeneration.

For purposes of this invention, the ECM material from any of the mammalian tissue consists of several basically inseparable layers broadly termed ECM material. For example, where it is thought that separating a basement membrane from the submucosa is considered to be very difficult, if not impossible, because the layers are thin and it is not possible to delaminate them from each other, the ECM material from that particular layer will probably necessarily contain some basement membrane with the submucosa.

According to the invention, the ECM compositions of the invention can also comprise ECM material from two or more mammalian sources. Thus, for example, the composition can comprise ECM material combinations from such sources as, for example, but not limited to, small intestine submucosa, liver basement membrane, stomach submucosa, urinary bladder submucosa, placental basement membrane, pancreatic basement membrane, large intestine submucosa, lung interstitial membrane, respiratory tract submucosa, heart ECM material, dermal matrix, and, in general, ECM material from any mammalian fetal tissue. The ECM material sources can also comprise different mammalian animals or an entirely different species of mammals.

The ECM composition can thus comprise ECM material from three mammalian tissue sources, four mammalian tissue sources, five mammalian tissue sources, six mammalian tissue sources, and conceivably up to ten or more tissue sources. The tissue sources can be from the same mammal (for example the same cow, the same pig, the same rodent, the same human, etc.), the same species of mammal (e.g. cow, pig, rodent, human), or different mammalian animals, but the same species, (e.g. cow 1 and cow 2, or pig 1 and pig 2), or different species of mammals (for example liver matrix from a pig, small intestine submucosa from a cow, and urinary bladder submucosa from a dog, all mixed together in the composition).

According to the invention, the ECM material can comprise mixed solid particulates. The ECM material can also be formed into a particulate and fluidized, as described in U.S. Pat. Nos. 5,275,826, 6,579,538 and 6,933,326, to form a mixed emulsion, mixed gel or mixed paste.

In some embodiments of the invention, the ECM compositions comprise sterilized acellular ECM compositions that are preferably formed by contemporaneously sterilizing and decellularizing an isolated ECM material.

Suitable methods for producing sterilized acellular ECM compositions are set forth in U.S. Pat. Nos. 7,108,832 and 8,034,288, and Co-Pending application Ser. Nos. 13/480,140, 12/707,427, 13/480,205, and 11/747,028; which are incorporated by reference herein in their entirety.

According to the invention, the liquid or semi-solid components of the ECM compositions (i.e. gels, emulsions or pastes) can comprise various concentrations. Preferably, the concentration of the liquid or semi-solid components of the ECM compositions are in the range of about 0.001 mg/ml to about 200 mg/ml. Suitable concentration ranges thus include, without limitation: about 5 mg/ml to about 150 mg/ml, about 10 mg/ml to about 125 mg/ml, about 25 mg/ml to about 100 mg/ml, about 20 mg/ml to about 75 mg/ml, about 25 mg/ml to about 60 mg/ml, about 30 mg/ml to about 50 mg/ml, and about 35 mg/ml to about 45 mg/ml and about 40 mg/ml. to about 42 mg/ml.

The noted concentration ranges are, however, merely exemplary and not intended to be exhaustive or limiting. It is understood that any value within any of the listed ranges is deemed a reasonable and useful value for a concentration of a liquid or semi-solid component of an ECM composition.

According to the invention, the dry particulate or reconstituted particulate that forms a gel emulsion or paste of the two ECM materials can also be mixed together in various proportions. For example, the particulates can comprise 50% of small intestine submucosa mixed with 50% of pancreatic basement membrane. The mixture can then similarly be fluidized by hydrating in a suitable buffer, such as saline.

According to the invention, the ECM compositions (and/or ECM core members) of the invention can further include one or more additional bioactive agents or components to aid in the treatment of damaged tissue and/or facilitate the tissue regenerative process.

In some embodiments, the bioactive agent(s) comprise a pharmacological agent or composition, which can comprise, without limitation, antibiotics or antifungal agents, anti-viral agents, anti-pain agents, anesthetics, analgesics, steroidal anti-inflammatories, non-steroidal anti-inflammatories, anti-neoplastics, anti-spasmodics, modulators of cell-extracellular matrix interactions, proteins, hormones, enzymes and enzyme inhibitors, anticoagulants and/or antithrombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

Suitable pharmacological agents and/or compositions thus include, without limitation, atropine, tropicamide, dexamethasone, dexamethasone phosphate, betamethasone, betamethasone phosphate, prednisolone, triamcinolone, triamcinolone acetonide, fluocinolone acetonide, anecortave acetate, budesonide, cyclosporine, FK-506, rapamycin, ruboxistaurin, midostaurin, flurbiprofen, suprofen, ketoprofen, diclofenac, ketorolac, nepafenac, lidocaine, neomycin, polymyxin b, bacitracin, gramicidin, gentamicin, oyxtetracycline, ciprofloxacin, ofloxacin, tobramycin, amikacin, vancomycin, cefazolin, ticarcillin, chloramphenicol, miconazole, itraconazole, trifluridine, vidarabine, ganciclovir, acyclovir, cidofovir, ara-amp, foscarnet, idoxuridine, adefovir dipivoxil, methotrexate, carboplatin, phenylephrine, epinephrine, dipivefrin, timolol, 6-hydroxydopamine, betaxolol, pilocarpine, carbachol, physostigmine, demecarium, dorzolamide, brinzolamide, latanoprost, sodium hyaluronate, insulin, verteporfin, pegaptanib, ranibizumab, and other antibodies, antineoplastics, Anti VGEFs, ciliary neurotrophic factor, brain-derived neurotrophic factor, bFGF, Caspase-1 inhibitors, Caspase-3 inhibitors, α-Adrenoceptors agonists, NMDA antagonists, Glial cell line-derived neurotrophic factors (GDNF), pigment epithelium-derived factor (PEDF), and NT-3, NT-4, NGF, IGF-2.

According to the invention, the amount of a pharmacological agent added to an ECM composition (and/or ECM core member) of the invention will, of course, vary from agent to agent. For example, in one embodiment, wherein the pharmacological agent comprises dicloflenac (Voltaren®), the amount of dicloflenac included in the ECM composition is preferably in the range of 10 μg-75 mg.

In some embodiments of the invention, the pharmacological agent specifically comprises one of the aforementioned anti-inflammatory agents.

According to the invention, the amount of an anti-inflammatory added to an ECM composition (and/or ECM core members) of the invention can similarly vary from anti-inflammatory to anti-inflammatory. For example, in one embodiment of the invention, wherein the pharmacological agent comprises ibuprofen (Advil®), the amount of ibuprofen included in the ECM composition is preferably in the range of 100 μg-200 mg.

In some embodiments of the invention, the bioactive agent comprises a statin, i.e. a HMG-CoA reductase inhibitor. According to the invention, suitable statins include, without limitation, atorvastatin (LIPITOR®), cerivastatin, fluvastatin (Lescol®), lovastatin (Mevacor®, Altocor®, Altoprev®), mevastatin, pitavastatin (Livalo®, Pitava®), pravastatin (Pravachol®, Selektine®, Lipostat®), rosuvastatin (Crestor®), and simvastatin (Zocor®, Lipex®). Several actives comprising a combination of a statin and another agent, such as ezetimbe/simvastatin (Vytorin®), are also suitable.

Applicant has found that the noted statins exhibit numerous beneficial properties that provide several beneficial biochemical actions or activities. Several significant properties and beneficial actions resulting therefrom are discussed in detail below. Additional properties and beneficial actions are set forth in Co-Pending application Ser. No. 13/373,569; which is incorporated by reference herein in its entirety.

### Anti-Inflammatory Properties/Actions

Statins have numerous favorable effects on vascular wall cells and the cardiovascular system. One specific example is that statins facilitate the reduction of the G-Protein-Coupled Receptor, thromboxane A2 (TXA₂), which lowers the platelet activation and aggregation, and augmentation of adhesion molecules and chemokines.

Statins further impact vascular wall cells and the cardiovascular system by blocking ras homilog gene family, member A (RhoA) activation. Blocking RhoA activation further impacts numerous systems, such as macrophage growth, tissue plasminogen activators (t-PA), plasminogen activator inhibitor type 1 (PAI-1), smooth muscle cell (SMC) proliferation, nitric oxide (NO) production, endothelins, and angiotensin receptors.

Macrophage growth reduced by blocking RhoA activation results in the reduction of matrix metalloprotinases (MMPs) and tissue factors (TF). Lowered MMPs also results in a lowered presence of thrombi, as the MMPs attach to ECM present in thrombi or damaged ECM at wound sites.

### Fibrinolysis Properties/Actions

Blocking RhoA activation also affects the presence of tissue plasminogen activators (t-PA) and plasminogen activator inhibitor type 1 (PAI-1), which is the principal inhibitor of fibrinolysis. With t-PA presence raised and PAI-1 diminished from the blocking of RhoA activation induced by statins, a reduced thrombotic effect is realized due to reduced opportunity for fibrin to form the polymeric mesh of a hemostatic plug.

### NO Regulation Properties/Actions

Blocking RhoA activation also affects the presence of Nitric Oxide (NO) in the cardiovascular system. NO contributes to vessel homeostasis by inhibiting vascular smooth muscle contraction and growth, platelet aggregation, and leukocyte adhesion to the endothelium.

### RhoA Activation Blocking Properties/Actions

The administration of statins can also enhance the presence of endothelins and angiotensin receptors. Endothelins and angiotensin receptors can also be affected by the subsequent blocking of RhoA activation associated with statin administration.

There are three isoforms of endothelins; ET-1, ET-2, and ET-3, with ET-1 being the isoform primarily affected by statins and RhoA activation blocking. Secretion of ET-1 from the endothelium signals vasoconstriction and influences local cellular growth and survival.

Angiotensin receptors are protein coupled receptors that are responsible for the signal transduction of the vasoconstricting stimulus of the main effector hormone angiotensin II. Angiotensin Receptor II Type I (AT-1) is the angiotensin receptor primarily affected by statin administration and RhoA activation blocking. AT-1 mediates vasocontraction, cardiac hypertrophy, vascular smooth muscle cell proliferation, inter alia.

### C-Reactive Protein Reduction Properties/Actions

C-Reactive Proteins (CRP) are also reduced by statins. CRPs are found in the blood; the levels of which deviate in response to differing levels of inflammation.

### Adhesion Molecule Reduction Properties/Actions

Statins also reduce the presence of adhesion molecules on the endothelium. Adhesion molecules are proteins that are located on the cell surface and are involved with inflammation and thrombin formation in vascular endothelial cells.

### Rac-1 Reduction Properties/Actions

The expression of Rac-1 is also reduced by statins. Rac-1 is a protein found in human cells, which plays a central role in endothelial cell migration, tubulogenesis, adhesion, and permeability. The decrease in the presence of Rac-1 also results in the decrease of reactive oxygen species (ROS).

According to the invention, the ECM material can include 10 mg or greater of a statin to achieve a higher concentration of the statin within a desired tissue, or 10 ug or less to achieve a lower concentration of the statin within a desired tissue.

According to the invention, the amount of a statin added to an ECM composition (and/or ECM core member) is preferably less than 20 mg, more preferably, less than approximately 10 mg.

In some embodiments of the invention, the ECM composition (and/or ECM core member) includes 100 ug-5 mg of a statin. In some embodiments of the invention, the ECM composition (and/or ECM core member) includes 500 ug-2 mg of a statin.

In some embodiments of the invention, the bioactive agent comprises chitosan or a derivative thereof. As also set forth in detail in Co-Pending application Ser. No. 13/573,569, chitosan also exhibits numerous beneficial properties that provide several beneficial biochemical actions or activities.

According to the invention, the amount of chitosan added to an ECM composition (and/or ECM core member) of the invention is preferably less than 50 ml, more preferably, less than approximately 20 ml.

In some embodiments of the invention, the chitosan is incorporated in a polymeric network, such as disclosed in U.S. Pub. Nos. 2008/0254104 and 2009/0062849, which are incorporated herein in their entirety.

In some embodiments of the invention, the bioactive agent comprises a cell. According to the invention, the cell can comprise, without limitation, a stem cell, such as, for example, a human embryonic stem cell, fetal cell, fetal cardiomyocyte, myofibroblast, mesenchymal stem cell, autotransplanted expanded cardiomyocyte, adipocyte, totipotent cell, pluripotent cell, blood stem cell, myoblast, adult stem cell, bone marrow cell, mesenchymal cell, embryonic stem cell, parenchymal cell, epithelial cell, endothelial cell, mesothelial cell, fibroblast, myofibroblast, osteoblast, chondrocyte, exogenous cell, endogenous cell, stem cell, hematopoetic stem cell, pluripotent stem cell, bone marrow-derived progenitor cell, progenitor cell, myocardial cell, skeletal cell, undifferentiated cell, multi-potent progenitor cell, unipotent progenitor cell, monocyte, cardiomyocyte, cardiac myoblast, skeletal myoblast, macrophage, capillary endothelial cell, xenogenic cell, and allogenic cell.

In some embodiments of the invention, the bioactive agent comprises a protein. According to the invention, the protein can comprise, without limitation, collagen, proteoglycan, glycosaminoglycan (GAG) chain, glycoprotein, cytokine, cell-surface associated protein, cell adhesion molecule (CAM), angiogenic growth factor, endothelial ligand, matrikine, matrix metalloprotease, cadherin, immunoglobin, fibril collagen, non-fibrillar collagen, basement membrane collagen, multiplexin, small-leucine rich proteoglycan, decorin, biglycan, fibromodulin, keratocan, lumican, epiphycan, heparan sulfate proteoglycan, perlecan, agrin, testican, syndecan, glypican, serglycin, selectin, lectican, aggrecan, versican, nuerocan, brevican, cytoplasmic domain-44 (CD44), macrophage stimulating factor, amyloid precursor protein, heparin, chondroitin sulfate B (deimatan sulfate), chondroitin sulfate A, heparan sulfate, hyaluronic acid, fibronectin (Fn), tenascin, elastin, fibrillin, laminin, nidogen/entactin, fibulin I, fibulin II, integrin, a transmembrane molecule, thrombospondin, osteopontin, and angiotensin converting enzyme (ACE).

In some embodiments of the invention, the bioactive agent comprises a growth factor. According to the invention, the growth factor can comprise, without limitation, a platelet derived growth factor (PDGF), epidermal growth factor (EGF), transforming growth factor-α (TGF-α), transforming growth factor-β (TGF-β), fibroblast growth factor-2 (FGF-2), basic fibroblast growth factor (bFGF), vascular epithelial growth factor (VEGF), hepatocyte growth factor (HGF), insulin-like growth factor (IGF), nerve growth factor (NGF), platelet derived growth factor (PDGF), tumor necrosis factor-α (TNA-α), and placental growth factor (PLGF).

In some embodiments of the invention, the ECM compositions (and/or ECM core members) specifically include a statin and chitosan. It has been found that the synergistic actions exhibited by the combination of a statin and chitosan significantly enhance the inducement of neovascularization, host tissue proliferation, bioremodeling, and regeneration of new tissue and associated structures (with site-specific structural and functional properties) when administered to damaged or diseased biological tissue.

According to the invention, the bioactive agents referenced herein can comprise any form. In some embodiments of the invention, the bioactive component or components, e.g. simvastatin and/or chitosan, comprise microcapsules that provide delayed delivery of the agent contained therein.

As indicated above, in some embodiments of the invention, the ECM based structures or “ECM nerve regeneration members” include an ECM core member having at least one ECM composition layer disposed thereon.

In some embodiments, the ECM core member has a tubular shape.

In some embodiments, the tubular shaped ECM core member includes a plurality of internal conduits.

In some embodiments, the ECM composition layer comprises an ECM composition coating. In some embodiments, the ECM composition layer comprises a plurality of ECM composition coatings.

According to the invention, various conventional means can be employed to coat the ECM composition on the outer surface of the ECM nerve regeneration members, including spray coating, dipping, etc.

In some embodiments, the ECM composition layer comprises an ECM composition sheet member. In some embodiments, the ECM composition layer comprises a plurality of ECM composition sheet members.

In some embodiments, the ECM composition layer comprises at least one ECM composition coating and at least one ECM composition sheet member.

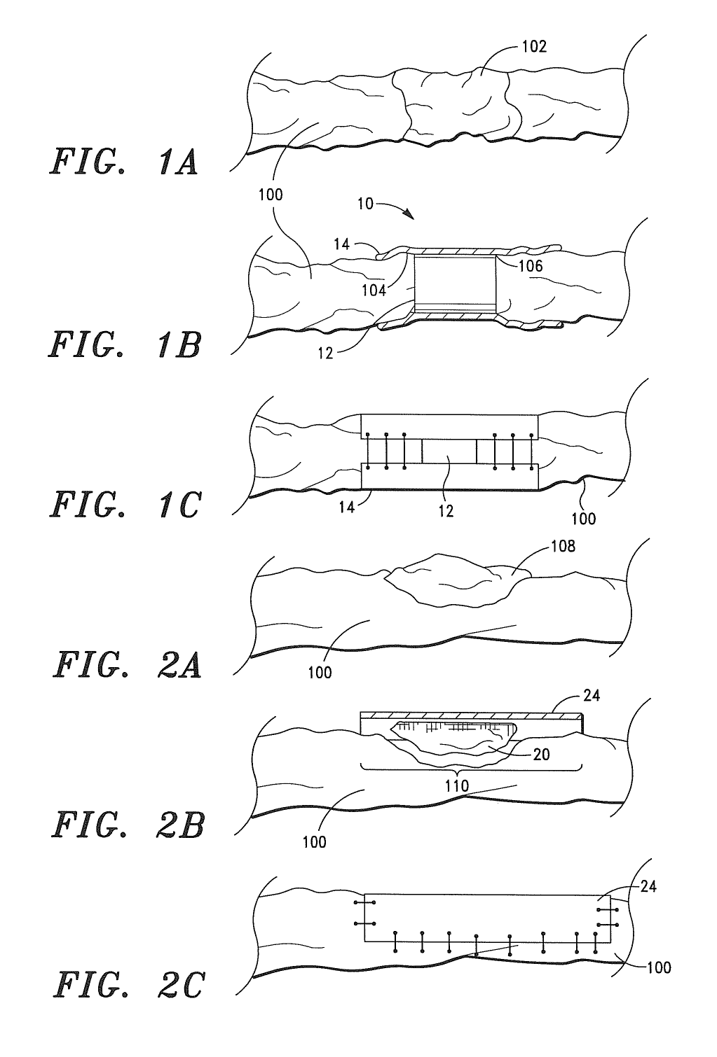

Referring now to FIG. 1B, there is shown one embodiment of an ECM nerve regeneration member of the invention. As illustrated in FIG. 1B, the ECM nerve regeneration member 10 includes a tubular ECM core member or structure 12 and an outer ECM composition layer, which, in the illustrated embodiment, comprises an ECM sheet 14. As illustrated in FIG. 1B and discussed in detail below, in some embodiments, the ECM sheet 14 is designed and configured to wrap around the ECM core structure 12 and at least a portion of the neural tissue 100.

As indicated above, the ECM core structure 12 and ECM sheet 14 are constructed of an ECM composition that includes at least one ECM material that is derived from one or more mammalian tissue sources, including the small intestine, large intestine, stomach, lung, liver, kidney, pancreas, placenta, heart, bladder, prostate, tissue surrounding growing enamel, tissue surrounding growing bone, and any fetal tissue from any mammalian organ, and methods for preparing same.

As also indicated above, the ECM compositions can further include one or more additional biologically active components to facilitate the treatment of damaged tissue and/or the tissue regenerative process, including one or more pharmacological agents or compositions, e.g., anti-inflammatory.

Referring now to FIG. 1A, in one embodiment of the invention, wherein a section of neural tissue 100 is fully resected and has had scar tissue 102 form, the scar tissue 102 is initially debrided and replaced with the ECM core structure 12 (see FIG. 1B). The ECM core structure 12 and the debrided neural tissue ends 104, 106 are then wrapped with the ECM composition sheet 14, as shown in FIG. 1C.

Referring now to FIGS. 2A-2C, for a section of neural tissue 100 that has not been fully resected, but presents with scar tissue 108, the scar tissue 108 is similarly debrided and replaced with an ECM core structure 20. The ECM core structure 20 and a region of the neural tissue 110 are then covered with an ECM composition sheet 24, as shown in FIG. 2C.

Referring now to FIG. 3, in a further embodiment of the invention, wherein the neural tissue 100 presents with a section of fibrosis 105, the section of fibrosis 105 is initially removed and debrided. An ECM composition sheet 34 is then placed over the debrided region and a section of neural tissue 110. After the ECM composition sheet 34 is attached to the neural tissue 100, an injectable (or emulsified) ECM composition 200 is injected into the space between the neural tissue 100 and ECM sheet 34.

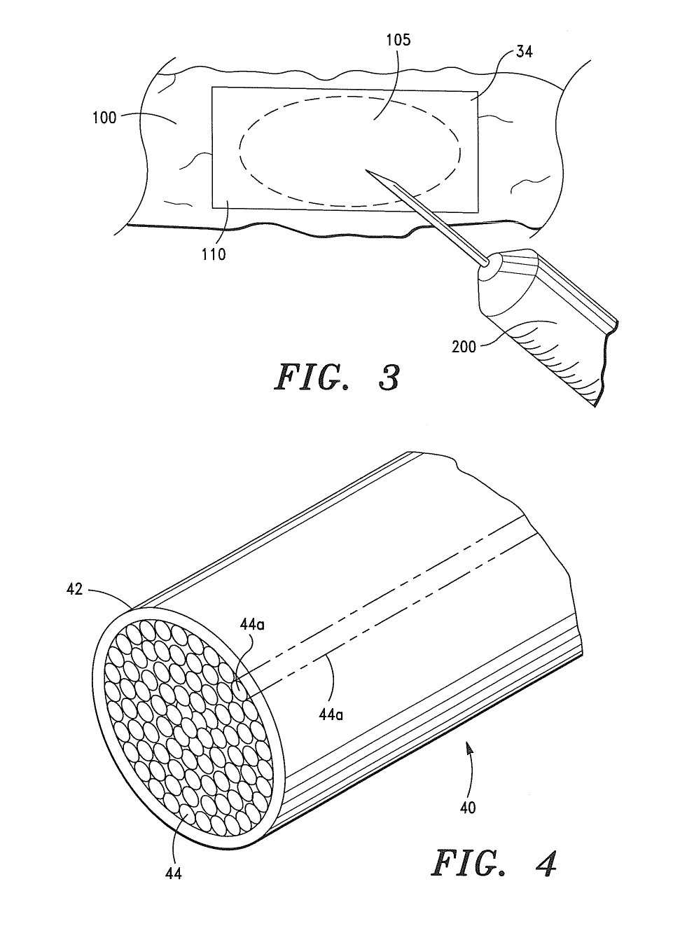

In further embodiments of the invention, wherein a neural pathway has undergone a full resection, an ECM nerve regeneration member of the invention can similarly be employed. Referring now to FIG. 4, in the noted embodiments, the ECM nerve regeneration member 40 includes an ECM core structure 42 having full-length conduits 44 that allow for augmentation of natural neural regeneration in the PNS.

According to the invention, there can be as little as two (2) full length conduits 44 to over one-hundred (100) conduits 44.

In some embodiments of the invention, the ECM nerve regeneration member can also include an outer ECM composition layer, such as an ECM coating or sheet.

According to the invention, upon deployment of an ECM nerve regeneration member of the invention in the damaged or resected neural pathways, modulated healing, including the regeneration of neural tissue and structures (with site-specific functional properties), is effectuated.

As will readily be appreciated by one having ordinary skill in the art, the present invention provides numerous advantages compared to prior art methods and systems for repairing damaged or diseased neural tissue. Among the advantages are the following:
The provision of extracellular matrix (ECM) based compositions, structures and methods that effectively suppress inhibitory nerve regeneration mechanisms and enhance neurotrophic nerve regeneration mechanisms.
The provision of ECM based compositions, structures and methods that induce modulated healing of damaged and/or diseased neural tissue.
The provision of ECM based compositions, structures and methods that induce regeneration of neural tissue and structures with site-specific functional properties.
The provision of ECM based compositions, structures and methods that modulate the inflammatory phase (e.g., platelet or fibrin deposition) at the beginning of the tissue healing process.
The provision of ECM based compositions, structures and methods that induce host tissue proliferation and bioremodeling, including neovascularization.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. A nerve regeneration device, comprising:
a support structure having an outer surface and a plurality of conduits extending therethrough, said support structure comprising a first extracellular matrix (ECM) material from a mammalian tissue source,
said support structure outer layer including at least a first layer comprising a first ECM composition, said first ECM composition including at least a second ECM material from a mammalian tissue source,
wherein, when said nerve regeneration device is deployed proximate damaged neural tissue, said nerve regeneration device induces modulated healing of said damaged tissue.

2. The nerve regeneration device of claim 1, wherein said first ECM composition layer comprises a first ECM composition coating.

3. The nerve regeneration device of claim 1, wherein said first ECM composition layer comprises a first ECM composition sheet member.

4. The nerve regeneration device of claim 2, wherein said support structure outer layer includes a second ECM composition layer, said second ECM composition layer comprising said first ECM composition.

5. The nerve regeneration device of claim 4, wherein said second ECM composition layer comprises a second ECM composition coating.

6. The nerve regeneration device of claim 4, wherein said second ECM composition layer comprises a second ECM composition sheet member.

7. The nerve regeneration device of claim 1, wherein said first ECM material is selected from the group consisting of small intestine submucosa (SIS), urinary bladder submucosa (UBS), urinary basement membrane (UBM), liver basement membrane (LBM), stomach submucosa (SS), mesothelial tissue, subcutaneous extracellular matrix, large intestine extracellular matrix, placental extracellular matrix, ornamentum extracellular matrix, heart extracellular matrix and lung extracellular matrix.

8. The nerve regeneration device of claim 7, wherein said first ECM material further includes at least a first biologically active agent.

9. The nerve regeneration device of claim 8, wherein said first biologically active agent comprises a growth factor selected from the group consisting of a platelet derived growth factor (PDGF), epidermal growth factor (EGF), transforming growth factor-α (TGF-α), transforming growth factor-β (TGF-β), fibroblast growth factor-2 (FGF-2), basic fibroblast growth factor (bFGF), vascular epithelial growth factor (VEGF), hepatocyte growth factor (HGF), insulin-like growth factor (IGF), nerve growth factor (NGF), platelet derived growth factor (PDGF), tumor necrosis factor-α (TNA-α), and placental growth factor (PLGF).

10. The nerve regeneration device of claim 8, wherein said first biologically active agent comprises a cell selected from the group consisting of a human embryonic stem cell, fetal cardiomyocyte, myofibroblast, mesenchymal stem cell, autotransplanted expanded cardiomyocytes, adipocyte, totipotent cell, pluripotent cell, blood stem cell, myoblast, adult stem cell, bone marrow cell, mesenchymal cell, embryonic stem cell, parenchymal cell, epithelial cell, endothelial cell, mesothelial cell, fibroblast, osteoblast, chondrocyte, exogenous cell, endogenous cell, hematopoietic stem cell, bone-marrow derived progenitor cell, myocardial cell, skeletal cell, fetal cell, undifferentiated cell, multi-potent progenitor cell, unipotent progenitor cell, monocyte, cardiac myoblast, skeletal myoblast, macrophage, capillary endothelial cell, xenogenic cell, allogenic cell and post-natal stem cell.

11. The nerve regeneration device of claim 8, wherein said first biologically active agent comprises an active agent selected from the group consisting of a collagen (types I-V), proteoglycans, glycosaminoglycans (GAGs), glycoproteins, cytokines, cell-surface associated proteins, cell adhesion molecules (CAMs), endothelial ligands, matrikines, cadherins, immuoglobins, fibril collagens, non-fibrallar collagens, basement membrane collagens, multiplexins, small-leucine rich proteoglycans, decorins, biglycans, fibromodulins, keratocans, lumicans, epiphycans, heparin sulfate proteoglycans, perlecans, agrins, testicans, syndecans, glypicans, serglycins, selectins, lecticans, aggrecans, versicans, neurocans, brevicans, cytoplasmic domain-44 (CD-44), macrophage stimulating factors, amyloid precursor proteins, heparins, chondroitin sulfate B (derrnatan sulfate), chondroitin sulfate A, heparin sulfates, hyaluronic acids, fibronectins, tenascins, elastins, fibrillins, laminins, nidogen/enactins, fibulin I, finulin II, integrins, transmembrane molecules, thrombospondins, ostepontins, and angiotensin converting enzymes (ACE).

12. The nerve regeneration device of claim 8, wherein said first biologically active agent comprises a HMG-CoA reductase inhibitor.

13. The nerve regeneration device of claim 12, wherein said HMG-CoA reductase inhibitor is selected from the group consisting of atorvastatin, cerivastatin, fluvastatin, lovastatin, mevastatin, pitavastatin, pravastatin, rosuvastatin and simvastatin.

14. The nerve regeneration device of claim 8, wherein said first biologically active agent comprises chitosan.

15. The nerve regeneration device of claim 8, wherein said first biologically active agent comprises a pharmacological agent.

16. The nerve regeneration device of claim 15, wherein said pharmacological agent is selected from the group consisting of antibiotics, antifungal agents, anti-viral agents, anti-pain agents, anesthetics, analgesics, steroidal anti-inflammatories, non-steroidal anti-inflammatories, anti-neoplastics, anti-spasmodics, modulators of cell-extracellular matrix interactions, proteins, hormones, enzymes and enzyme inhibitors, anticoagulants, antithrombic agents, DNA, RNA, modified DNA and RNA, NSAIDs, inhibitors of DNA, polypeptides, oligonucleotides, polynucleotides, nucleoproteins, and vasodilating agents.

17. The nerve regeneration device of claim 1, wherein said second ECM material is selected from the group consisting of small intestine submucosa (SIS), urinary bladder submucosa (UBS), urinary basement membrane (UBM), liver basement membrane (LBM), stomach submucosa (SS), mesothelial tissue, subcutaneous extracellular matrix, large intestine extracellular matrix, placental extracellular matrix, ornamentum extracellular matrix, heart extracellular matrix and lung extracellular matrix.

18. The nerve regeneration device of claim 17, wherein said second ECM material further includes at least a second biologically active agent.

19. The nerve regeneration device of claim 18, wherein said second biologically active agent comprises a growth factor selected from the group consisting of a platelet derived growth factor (PDGF), epidermal growth factor (EGF), transforming growth factor-α (TGF-α), transforming growth factor-β (TGF-β), fibroblast growth factor-2 (FGF-2), basic fibroblast growth factor (bFGF), vascular epithelial growth factor (VEGF), hepatocyte growth factor (HGF), insulin-like growth factor (IGF), nerve growth factor (NGF), platelet derived growth factor (PDGF), tumor necrosis factor-α (TNA-α), and placental growth factor (PLGF).

20. The nerve regeneration device of claim 18, wherein said second biologically active agent comprises a cell selected from the group consisting of a human embryonic stem cell, fetal cardiomyocyte, myofibroblast, mesenchymal stem cell, autotransplanted expanded cardiomyocytes, adipocyte, totipotent cell, pluripotent cell, blood stem cell, myoblast, adult stem cell, bone marrow cell, mesenchymal cell, embryonic stem cell, parenchymal cell, epithelial cell, endothelial cell, mesothelial cell, fibroblast, osteoblast, chondrocyte, exogenous cell, endogenous cell, hematopoietic stem cell, bone-marrow derived progenitor cell, myocardial cell, skeletal cell, fetal cell, undifferentiated cell, multi-potent progenitor cell, unipotent progenitor cell, monocyte, cardiac myoblast, skeletal myoblast, macrophage, capillary endothelial cell, xenogenic cell, allogenic cell and post-natal stem cell.

21. The nerve regeneration device of claim 18, wherein said second biologically active agent comprises an active agent selected from the group consisting of a collagen (types I-V), proteoglycans, glycosaminoglycans (GAGs), glycoproteins, cytokines, cell-surface associated proteins, cell adhesion molecules (CAMs), endothelial ligands, matrikines, cadherins, immuoglobins, fibril collagens, non-fibrallar collagens, basement membrane collagens, multiplexins, small-leucine rich proteoglycans, decorins, biglycans, fibromodulins, keratocans, lumicans, epiphycans, heparin sulfate proteoglycans, perlecans, agrins, testicans, syndecans, glypicans, serglycins, selectins, lecticans, aggrecans, versicans, neurocans, brevicans, cytoplasmic domain-44 (CD-44), macrophage stimulating factors, amyloid precursor proteins, heparins, chondroitin sulfate B (dermatan sulfate), chondroitin sulfate A, heparin sulfates, hyaluronic acids, fibronectins, tenascins, elastins, fibrillins, laminins, nidogen/enactins, fibulin I, finulin II, integrins, transmembrane molecules, thrombospondins, ostepontins, and angiotensin converting enzymes (ACE).

22. The nerve regeneration device of claim 18, wherein said second biologically active agent comprises a HMG-CoA reductase inhibitor.

23. The nerve regeneration device of claim 22, wherein said HMG-CoA reductase inhibitor is selected from the group consisting of atorvastatin, cerivastatin, fluvastatin, lovastatin, mevastatin, pitavastatin, pravastatin, rosuvastatin and simvastatin.

24. The nerve regeneration device of claim 18, wherein said second biologically active agent comprises chitosan.

25. The nerve regeneration device of claim 18, wherein said second biologically active agent comprises a pharmacological agent.

26. The nerve regeneration device of claim 25, wherein said pharmacological agent is selected from the group consisting of antibiotics, antifungal agents, anti-viral agents, anti-pain agents, anesthetics, analgesics, steroidal anti-inflammatories, non-steroidal anti-inflammatories, anti-neoplastics, anti-spasmodics, modulators of cell-extracellular matrix interactions, proteins, hormones, enzymes and enzyme inhibitors, anticoagulants, antithrombic agents, DNA, RNA, modified DNA and RNA, NSAIDs, inhibitors of DNA, polypeptides, oligonucleotides, polynucleotides, nucleoproteins, and vasodilating agents.

27. A method of promoting regeneration of neural tissue in a mammal comprising directly contacting damaged neural tissue with an ECM composition including an ECM material from a mammalian tissue source, wherein, said ECM composition induces modulated healing and, thereby, regeneration of said damaged tissue.

28. The method of claim 27, wherein said ECM material is selected from the group consisting of small intestine submucosa (SIS), urinary bladder submucosa (UBS), urinary basement membrane (UBM), liver basement membrane (LBM), stomach submucosa (SS), mesothelial tissue, subcutaneous extracellular matrix, large intestine extracellular matrix, placental extracellular matrix, ornamentum extracellular matrix, heart extracellular matrix and lung extracellular matrix.

29. The method of claim 28, wherein said ECM material further includes at least one supplemental biologically active agent.

30. The method of claim 29, wherein said biologically active agent comprises a growth factor selected from the group consisting of a platelet derived growth factor (PDGF), epidermal growth factor (EGF), transforming growth factor-α (TGF-α), transforming growth factor-β (TGF-β), fibroblast growth factor-2 (FGF-2), basic fibroblast growth factor (bFGF), vascular epithelial growth factor (VEGF), hepatocyte growth factor (HGF), insulin-like growth factor (IGF), nerve growth factor (NGF), platelet derived growth factor (PDGF), tumor necrosis factor-α (TNA-α), and placental growth factor (PLGF).

31. The method of claim 29, wherein said biologically active agent comprises a cell selected from the group consisting of a human embryonic stem cell, fetal cardiomyocyte, myofibroblast, mesenchymal stem cell, autotransplanted expanded cardiomyocytes, adipocyte, totipotent cell, pluripotent cell, blood stem cell, myoblast, adult stem cell, bone marrow cell, mesenchymal cell, embryonic stem cell, parenchymal cell, epithelial cell, endothelial cell, mesothelial cell, fibroblast, osteoblast, chondrocyte, exogenous cell, endogenous cell, hematopoietic stem cell, bone-marrow derived progenitor cell, myocardial cell, skeletal cell, fetal cell, undifferentiated cell, multi-potent progenitor cell, unipotent progenitor cell, monocyte, cardiac myoblast, skeletal myoblast, macrophage, capillary endothelial cell, xenogenic cell, allogenic cell and post-natal stem cell.

32. The method of claim 29, wherein said biologically active agent comprises an active agent selected from the group consisting of a collagen (types I-V), proteoglycans, glycosaminoglycans (GAGS), glycoproteins, cytokines, cell-surface associated proteins, cell adhesion molecules (CAMs), endothelial ligands, matrikines, cadherins, immuoglobins, fibril collagens, non-fibrallar collagens, basement membrane collagens, multiplexins, small-leucine rich proteoglycans, decorins, biglycans, fibromodulins, keratocans, lumicans, epiphycans, heparin sulfate proteoglycans, perlecans, agrins, testicans, syndecans, glypicans, serglycins, selectins, lecticans, aggrecans, versicans, neurocans, brevicans, cytoplasmic domain-44 (CD-44), macrophage stimulating factors, amyloid precursor proteins, heparins, chondroitin sulfate B (dermatan sulfate), chondroitin sulfate A, heparin sulfates, hyaluronic acids, fibronectins, tenascins, elastins, fibrillins, laminins, nidogen/enactins, fibulin I, finulin II, integrins, transmembrane molecules, thrombospondins, ostepontins, and angiotensin converting enzymes (ACE).

33. The method of claim 29, wherein said biologically active agent comprises a HMG-CoA reductase inhibitor.

34. The method of claim 33, wherein said HMG-CoA reductase inhibitor is selected from the group consisting of atorvastatin, cerivastatin, fluvastatin, lovastatin, mevastatin, pitavastatin, pravastatin, rosuvastatin and simvastatin.

35. The method of claim 29, wherein said biologically active agent comprises chitosan.

36. The method of claim 29, wherein said biologically active agent comprises a pharmacological agent.

37. The method of claim 36, wherein said pharmacological agent is selected from the group consisting of antibiotics, antifungal agents, anti-viral agents, anti-pain agents, anesthetics, analgesics, steroidal anti-inflammatories, non-steroidal anti-inflammatories, anti-neoplastics, anti-spasmodics, modulators of cell-extracellular matrix interactions, proteins, hormones, enzymes and enzyme inhibitors, anticoagulants, antithrombic agents, DNA, RNA, modified DNA and RNA, NSAIDs, inhibitors of DNA, polypeptides, oligonucleotides, polynucleotides, nucleoproteins, and vasodilating agents.
